(19) United States
(12) Patent Application Publication
(10) Pub. No.: US 2012/0277577 A1
(43) Pub. Date: Nov. 1, 2012

(54) BODY CAVITY ULTRASONIC PROBE AND ULTRASONIC DIAGNOSIS APPARATUS

(75) Inventors: Takashi OGAWA, Nasushiobara-shi (JP); Susumu HIKI, OTAWARA-SHI (JP); Yutaka OONUKI, OTAWARA-SHI (JP); Takashi KUBOTA, OTAWARA-SHI (JP)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/545,665

(22) Filed: Jul. 10, 2012

Related U.S. Application Data
(63) Continuation of application No. 12/567,391, filed on Sep. 25, 2009, now abandoned.

(30) Foreign Application Priority Data
Sep. 29, 2008 (JP) 2008-251126

(51) Int. Cl. A61B 8/12; A61B 6/12

(52) U.S. Cl. 600/424; 600/447

(57) ABSTRACT

A body cavity probe which scans an object with ultrasonic waves includes a probe main body, and a linear array constituted by a plurality of piezoelectric elements and a convex array constituted by a plurality of piezoelectric elements, which are provided in the probe main body to transmit ultrasonic waves to the object and receive an echo signal from the object. The linear array and the convex array have different array shapes. The respective array surfaces are included in the same plane.

### CROSS-REFERENCE TO RELATED APPLICATIONS

This application is based upon and claims the benefit of priority from prior Japanese Patent Application No. 2008-251126, filed Sep. 29, 2008, the entire contents of which are incorporated herein by reference.

### BACKGROUND OF THE INVENTION

1. Field of the Invention

The present invention relates to a body cavity ultrasonic probe and ultrasonic diagnosis apparatus which acquire ultrasonic images associated with an object by scanning the object with ultrasonic waves.

2. Description of the Related Art

Recently, an ultrasonic guided puncture operation has been developed, which allows to insert a needle into a lesion such as a tumor while checking the inside of the object with ultrasonic images. The ultrasonic guided puncture operation is performed to display a needle and a lesion on an ultrasonic image in real time, and hence has dramatically improved the accuracy and safety of puncture.

Depending on the position of a lesion, a doctor sometimes inserts an ultrasonic probe into a body cavity such as a rectum, vagina, or esophagus and inserts a needle into the lesion from inside the body cavity while checking the lesion from inside the body cavity. This operation therefore requires an ultrasonic probe called a body cavity probe like that disclosed in, for example, Jpn. Pat. Appln. KOKAI Publication No. 11-76242, which can be inserted into a body cavity.

A body cavity probe includes a rod-like probe main body to be inserted into a body cavity and a transducer array provided in the probe main body. Body cavity probes vary in type depending on the types and positions of transducer arrays. For example, such probes include a type having a linear array placed on a side surface of the probe main body, a type having a convex array placed on the distal end of the probe main body, and a type (biplane probe) which is formed by combining the two types to scan cross-sections crossing each other. Some probes use convex arrays instead of linear arrays.

A puncture operation using a conventional body cavity probe, however, has the following problems. A conventional body cavity probe having ultrasonic transducers arrayed on only a side surface or distal end of a probe main body can only visualize either a predetermined region on the side surface side or a predetermined region on the distal end side. Even a biplane probe cannot simultaneously drive the transducer arrays arranged on the side surface and the distal end, and hence can only visualize either a predetermined region on the side surface side or a predetermined region on the distal end side. This increases an area of the insertion path of a puncture needle which cannot be visually checked with an ultrasonic image (an area of the insertion path of the puncture needle which is not visualized by an ultrasonic image) (to be referred to as a “blind area” hereinafter). The existence of a blood vessel or the like in a blind area will hinder the insertion of a puncture needle. This may make it impossible to smoothly perform an ultrasonic guided puncture operation.

### BRIEF SUMMARY OF THE INVENTION

The present invention provides a body cavity ultrasonic probe and ultrasonic diagnosis apparatus which can reduce a blind area of the insertion path of a puncture needle which cannot be visually recognized by an ultrasonic image in an ultrasonic guided puncture operation as compared with the prior art.

According to an aspect of the present invention, there is provided a body cavity ultrasonic probe which comprises: a substantially cylindrical insertion portion to be inserted into a body cavity of an object; and a plurality of ultrasonic transducers which are continuously arrayed from a side surface of the insertion portion to a distal end thereof so as to form one of a two-dimensional area or a three-dimensional area extending from the side surface of the insertion portion to the distal end as an ultrasonic scanning area.

According to another aspect of the present invention, there is provided an ultrasonic diagnosis apparatus which comprises: a body cavity ultrasonic probe including a substantially cylindrical insertion portion to be inserted into a body cavity of an object, and a plurality of ultrasonic transducers which are continuously arrayed from a side surface of the insertion portion to a distal end thereof so as to form one of a two-dimensional area or a three-dimensional area extending from the side surface of the insertion portion to the distal end as an ultrasonic scanning area; an ultrasonic transmission/reception unit which acquires an echo signal by transmitting an ultrasonic wave to the ultrasonic scanning area through the plurality of ultrasonic transducers and receiving a reflected wave from the ultrasonic scanning area through the plurality of ultrasonic transducers; an image generating unit which generates an ultrasonic image associated with the ultrasonic scanning area by using the echo signal; and a display unit which displays an ultrasonic image associated with the ultrasonic scanning area.

### BRIEF DESCRIPTION OF THE SEVERAL VIEWS OF THE DRAWING

FIG. 1 is a perspective view of an ultrasonic diagnosis apparatus according to an embodiment of the present invention;

FIG. 2 is a schematic view showing a controller according to the embodiment of the present invention;

FIG. 3 is a schematic view of a body cavity probe according to the embodiment of the present invention;

FIG. 4 is a perspective view of a probe main body according to the embodiment of the present invention;

FIG. 5 is a schematic view of a transmission/reception area formed by the body cavity probe according to the embodiment of the present invention;

FIG. 6 is a schematic view for explaining how an ultrasonic guided puncture operation is executed by the body cavity probe according to the embodiment of the present invention; and

FIG. 7 is a schematic view showing an ultrasonic image when a needle reaches a lesion in the embodiment of the present invention.

### DETAILED DESCRIPTION OF THE INVENTION

An embodiment of the present invention will be described in detail below with reference to the views of the accompanying drawing.

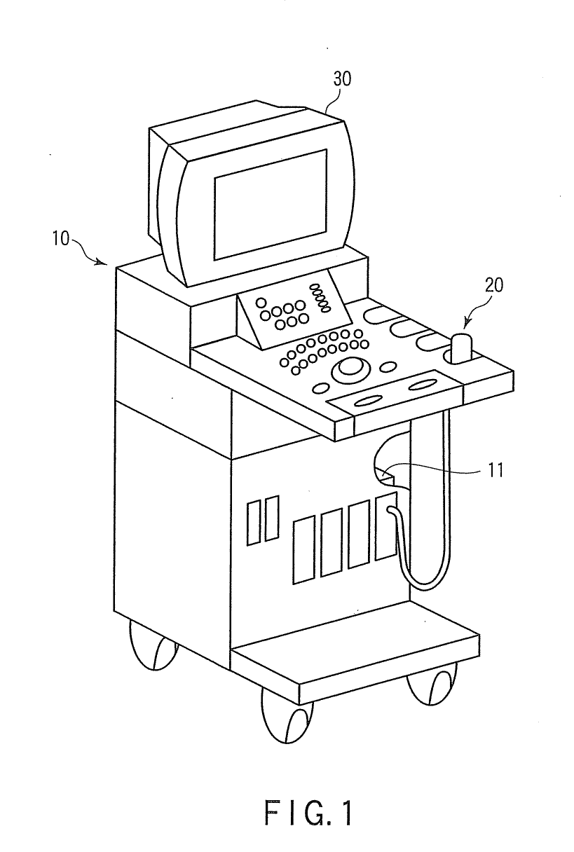

FIG. 1 is a perspective view of an ultrasonic diagnosis apparatus according to the first embodiment.

As shown FIG. 1, the ultrasonic diagnosis apparatus presents the internal state of an object P as an ultrasonic image I by using ultrasonic waves, and includes an apparatus body 10, a body cavity probe (ultrasonic probe) 20, and a monitor 30. The apparatus body 10 is configured to be movable using casters, and incorporates a controller 11 which executes various control operations and processes and the like.

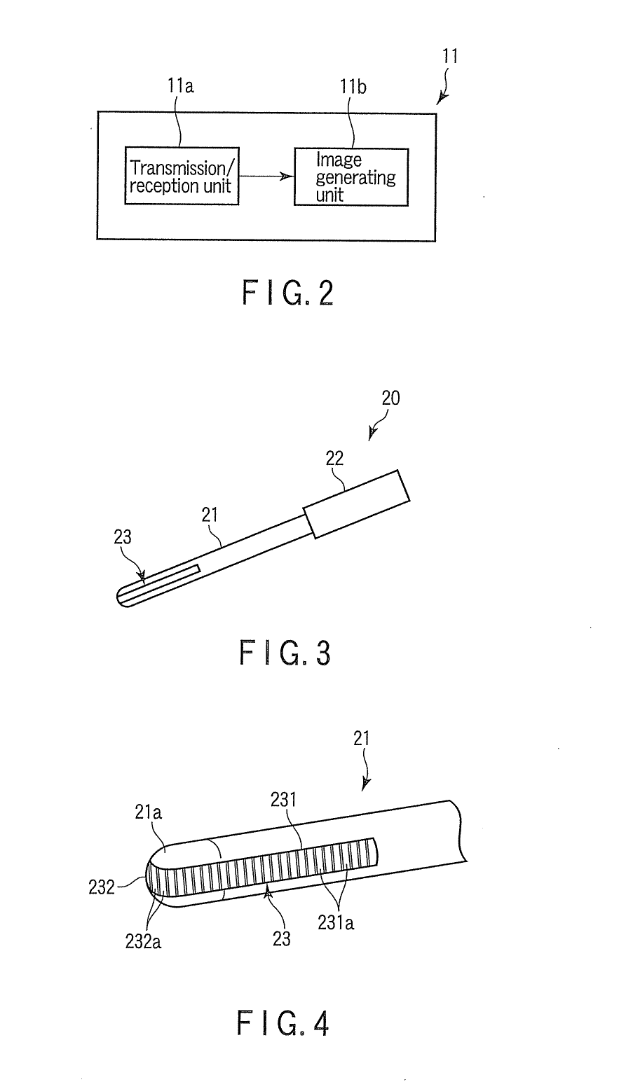

FIG. 2 is a schematic view of the controller 11 according to this embodiment.

As shown in FIG. 2, the controller 11 includes a transmission/reception unit (delay means) 11a and an image generating unit (image generating means) 11b. The transmission/reception unit 11a causes the body cavity probe 20 to execute transmission of an ultrasonic wave and reception of an echo signal. In addition, the transmission/reception unit 11a performs delay control on transmission of an ultrasonic wave and reception of an echo signal, as deeded. The image generating unit 11b generates the ultrasonic image I based on an echo signal from the transmission/reception unit 11a.

FIG. 3 is a schematic view of the body cavity probe 20 in this embodiment.

As shown in FIG. 3, the body cavity probe 20 includes a probe main body 21 to be inserted into a body cavity C such as a rectum, vagina, or esophagus and a grip portion 22 to be gripped by an operator.

FIG. 4 is a perspective view of the probe main body 21 in this embodiment.

As shown in FIG. 4, the probe main body 21 has a shape of a thin round bar, with a hemispherical portion 21a for prevention of damage to a living body being formed on the distal end of the probe main body. The probe main body 21 includes a transducer array 23 for the transmission/reception of ultrasonic waves to/from the object P.

The transducer array 23 is a so-called 1D array, and includes a linear array 231 provided on a side surface of the probe main body 21 and a convex array 232 provided on the distal end of the probe main body 21. Note that a convex array having a very small curvature is sometimes used in place of the linear array 231.

The linear array 231 includes a plurality of piezoelectric elements 231a arranged along the axis of the probe main body 21. Note that the intervals between the piezoelectric elements 231a are about 150 μm. The convex array 232 includes a plurality of piezoelectric elements 232a arranged along the hemispherical portion 21a of the probe main body 21. Note that the intervals between the piezoelectric elements 232a are about 100 μm.

The piezoelectric elements 231a included in the linear array 231 and the piezoelectric elements 232a included in the convex array 232 are all arranged in the same plane. Therefore, the linear array 231 and the convex array 232 can scan the object P with ultrasonic waves in the same plane.

The linear array 231 is placed near the convex array 232. With this arrangement, all the piezoelectric elements 231a and 232a included in the linear array 231 and the convex array 232 are continuously arranged from the linear array 231 to the convex array 232.

Although not shown in FIGS. 3 and 4, the body cavity probe 20 has a support unit for supporting a puncture needle while guiding its insertion direction to a predetermined path. This support unit supports a puncture needle such that the path of the puncture needle is included in the ultrasonic scanning plane formed by ultrasonic scanning by the linear array 231 and the convex array 232.

### (Generation of Beam)

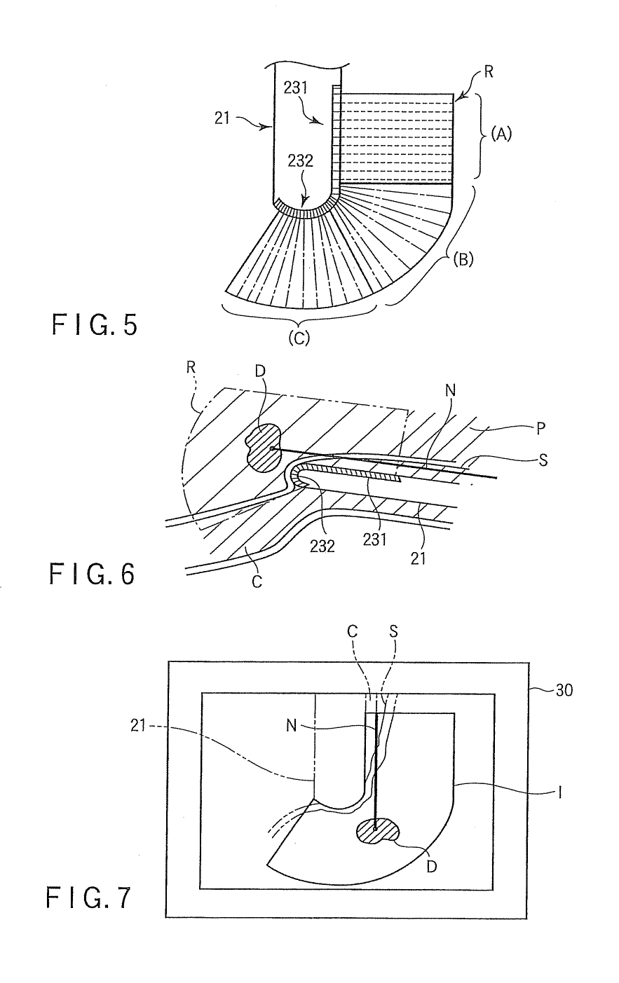

FIG. 5 is a schematic view of a transmission/reception range R formed by the body cavity probe 20 according to this embodiment. Referring to FIG. 5, the dotted lines, one-dot dashed lines, and two-dot dashed lines respectively express beams.

As shown in FIG. 5, the body cavity probe 20 forms the transmission/reception range R extending from the front surface of the linear array 231 to the front surface of the convex array 232. The transmission/reception range R includes a first area (A) formed by the piezoelectric elements 231a included in the linear array 231, a second area (B) formed by the piezoelectric elements 231a included in the linear array 231 and the piezoelectric elements 232a included in the convex array 232, and a third area (C) formed by the piezoelectric elements 232a included in the convex array 232.

Each beam formed in the first area (A) is formed by electronic scanning using a general linear array. The respective beams are therefore formed at almost right angles to the front surfaces of the piezoelectric elements 231a, and are arrayed almost parallel as a whole.

Each beam formed in the third area (C) is formed by electronic scanning using a general convex array. The respective beams are therefore formed at almost right angles to the front surfaces of the piezoelectric elements 232a, and are arrayed radially as a whole.

Each beam formed in the second area (B) is formed by electronic scanning using a general linear array and a general convex array. Note that the aperture widths are controlled to make the diameters of each beam on the two sides equal to each other. This makes it possible to form beams at desired positions even if the width of the piezoelectric elements 231a included in the linear array 231 differs from that of the piezoelectric elements 232a included in the convex array 232. Note that the focuses of the respective beams are respectively set on scanning lines by delay control.

### (Ultrasonic Guided Puncture Operation)

FIG. 6 is a schematic view for explaining how an ultrasonic guided puncture operation is executed by the body cavity probe 20 according to this embodiment.

As shown in FIG. 6, first of all, the probe main body 21 of the body cavity probe 20 is inserted into the body cavity C of the object P. When the linear array 231 and convex array 232 of the body cavity probe 20 reach a desired position on the object P, the probe starts transmission/reception of ultrasonic waves, and the monitor 30 displays the ultrasonic image I.

A needle N is then inserted into the body cavity C of the object P. It should be noted that the needle N is inserted parallel to the axis of the probe main body 21 at the front surface of the linear array 231. As the needle N proceeds, the needle point reaches the ultrasonic transmission/reception range R. This state is depicted on the ultrasonic image I. As the needle N further proceeds, the needle point is inserted into the object P from a surface S. When the needle point reaches a lesion D, an operation such as aspiration or cauterization is executed.

When the operation is complete, the needle N is pulled out. As the needle N recedes, the needle point is pulled off the surface S of the object P and reaches the body cavity C. As the needle N further recedes, the needle point is pulled off the ultrasonic transmission/reception range R. As a result, the state of the needle point disappears from the ultrasonic image I.

FIG. 7 is a schematic view showing the ultrasonic image I when the needle N reaches the lesion D in this embodiment.

As shown in FIG. 7, it is possible to depict a portion, of the needle N inserted into the object P, which extends from the side surface of the probe main body 21 to its distal end by using the ultrasonic image I. This greatly reduces the blind area of the puncture needle as compared with the prior art.

### (Operation in Embodiment)

In this embodiment, the body cavity probe 20 includes the linear array 231 on the side surface of the probe main body 21, and the convex array 232 on the distal end of the probe main body 21. The piezoelectric elements 231a included in the linear array 231 and the piezoelectric elements 232a included in the convex array 232 are arrayed in the same plane and can scan the same plane of the object P with ultrasonic waves. The image generating unit 11b generates each frame of the ultrasonic image I by using an echo signal from the linear array 231 and an echo signal from the convex array 232.

If the needle N inserted into the body cavity C exists at the front surface of the linear array 231, it is possible to depict a portion, of the needle N inserted into the object P, which extends from the side surface of the probe main body 21 to its distal end by depicting it using the ultrasonic image I. This makes it possible to greatly reduce the blind area of the puncture needle as compared with the prior art. It is also possible to visually recognize a path until the needle N reaches the lesion with an ultrasonic image. As a consequence, the accuracy and safety of the ultrasonic guided puncture operation can be improved.

Executing the puncture operation using the body cavity probe 20 can depict a wide area extending from the side surface of the probe main body 21 to its distal end by using the ultrasonic image I without performing image switching like the case of a biplane probe. It is therefore possible to improve the accuracy and safety of an ultrasonic guided puncture operation while reducing the operation load on a technician during the puncture operation.

In this embodiment, the linear array 231 and the convex array 232 are arranged such that all the piezoelectric elements 231a and 232a included in them are continuously arranged from the linear array 231 to the convex array 232.

For this reason, there is no joint in the boundary between the image area generated by the linear array 231 and the image area generated by the convex array 232, and hence the image quality of the ultrasonic image I does not deteriorate.

In this embodiment, the position of each beam formed in the second area (B) is adjusted by controlling the aperture width. For this reason, even if the width and intervals of the piezoelectric elements 231a included in the linear array 231 differ from those of the piezoelectric elements 232a included in the convex array 232, each beam is formed at an accurate position.

In this embodiment, the focuses of ultrasonic waves to be transmitted/received are set on the respective scanning lines by delay control using the transmission/reception unit 11a. This greatly improves the image quality of the ultrasonic image I.

Note that this embodiment uses the linear array 231 and the convex array 232. However, the present invention is not limited to this. That is, it is possible to use a convex array having a very small curvature instead of the linear array 231. Furthermore, it is possible to use a linear array instead of the convex array 232. In the latter case, it is preferable to perform control to form an ultrasonic scanning plane continuous from the side surface of the probe main body 21 to its distal end by executing oblique scanning to a part area or a whole area corresponding to the area (B) in FIG. 5, and sector scanning at the distal end of the probe main body 21.

Different ultrasonic transmission/reception conditions may be set for the linear array 231 and the convex array 232. For example, the display depth of the linear array 231 may be smaller than that of the convex array 232. Even if the display depth of the linear array 231 is small to some extent, since the needle N is inserted in a portion very close to the body cavity probe 20, the state of the needle N can be depicted on the ultrasonic image I without any problem. In addition, as the display depth of the linear array 231 decreases, the frame rate increases, leading to accurate depiction of the proceeding state of the needle N.

In this embodiment, all the beams formed by the linear array 231 are almost perpendicular to the linear array 231. However, the present invention is not limited to this. For example, the beams formed on the two ends of the linear array 231 can be tilted outward by oblique scanning. Note that oblique scanning is to tilt the direction of a beam by delay control. If the linear array 231 can form beams in the entire second area (B), there is no need to form beams in the second area (B) by using the piezoelectric elements 231a of the linear array 231 and the piezoelectric elements 232a of the convex array 232. This makes it unnecessary to perform aperture control in the first embodiment, and hence can acquire the desired ultrasonic image I by using only an existing scanning scheme.

The above embodiment has exemplified the case in which a plurality of ultrasonic transducers are arrayed to form two different types of array shapes, namely a linear array and a convex array. However, the present invention is not limited to this, and it is possible to array a plurality of ultrasonic transducers to form two or more different types of array shapes as needed. In addition, a plurality of ultrasonic transducers are arrayed in a line from the side surface of the probe main body 21 to its distal end. However, a plurality of ultrasonic transducers may be arranged in a plurality of arrays from the side surface of the probe main body 21 to its distal end. This arrangement can scan a three-dimensional area from the side surface of the probe main body 21 to its distal end with ultrasonic waves.

Note that the present invention is not limited to the above embodiment, and constituent elements can be variously modified and embodied at the execution stage within the spirit and scope of the invention. Various inventions can be formed by proper combinations of a plurality of constituent elements disclosed in the above embodiments. For example, several constituent elements may be omitted from all the constituent elements in each embodiment. In addition, constituent elements of the different embodiments may be combined as needed.

1. (canceled)

2. (canceled)

3. (canceled)

4. (canceled)

5. (canceled)

6. (canceled)

7. (canceled)

8. (canceled)

9. (canceled)

10. An ultrasonic diagnosis apparatus comprising:
a body cavity ultrasonic probe including a substantially cylindrical insertion portion to be inserted into a body cavity of an object, and a plurality of ultrasonic transducers which are continuously arrayed from a side surface of the insertion portion to a distal end thereof so as to form one of a two-dimensional area or a three-dimensional area extending from the side surface of the insertion portion to the distal end as an ultrasonic scanning area;
an ultrasonic transmission/reception unit which acquires an echo signal by transmitting an ultrasonic wave to the ultrasonic scanning area through the plurality of the ultrasonic transducers and receiving a reflected wave from the ultrasonic scanning area through the plurality of the ultrasonic transducers;
an image generating unit which generates an ultrasonic image associated with the ultrasonic scanning area by using the echo signal; and
a display unit which displays an ultrasonic image associated with the ultrasonic scanning area,
wherein the plurality of the ultrasonic transducers form a linear array portion on the side surface of the insertion portion, and a convex array portion on the distal end of the insertion portion; and
the ultrasonic transmission/reception unit controls a delay time for each of the ultrasonic transducers at the transmission and a delay time for each of the ultrasonic transducers at the reception so as to exert sector scanning at the convex array portion for forming a sector scanning area and oblique scanning at the linear array portion for forming an oblique scanning area toward the sector scanning area.

11. (canceled)

12. (canceled)

13. The apparatus according to claim 10, wherein the linear array portion on the side surface and the convex array portion on the distal end are formed from the side surface of the insertion portion to the distal end.

14. (canceled)

15. (canceled)

16. (canceled)

17. (canceled)

18. The apparatus according to claim 10, wherein an array direction of the plurality of the ultrasonic transducers corresponds to an insertion path direction in which a puncture needle is inserted into the object.

19. The apparatus according to claim 10, further comprising a support unit which supports a puncture needle such that an insertion path of the puncture needle inserted into the object is included in the ultrasonic scanning area.

20. The apparatus according to claim 10, wherein the ultrasonic transmission/reception unit controls a delay time for each of the ultrasonic transducers at the transmission and a delay time for said each ultrasonic transducer at the reception so as to make the ultrasonic scanning area be continuous from the side surface of the ultrasonic scanning area to the distal end.

21. The apparatus according to claim 10, wherein the ultrasonic transmission/reception unit controls a delay time for said each ultrasonic transducer at the transmission and a delay time for each of the ultrasonic transducers at the reception so as to make the ultrasonic scanning area continuous from the sector scanning area to the oblique scanning area.
